(19) United States
(12) Patent Application Publication
(10) Pub. No.: US 2016/0010081 A1
(43) Pub. Date: Jan. 14, 2016

(54) SYSTEMS, METHODS AND KITS FOR EXTRACTING NUCLEIC ACID FROM DIGESTIVE FLUID

(71) Applicant: Exact Sciences Corporation, Madison, WI (US)

(72) Inventors: Hatim T. Allawi, Middleton, WI (US); Graham P. Lidgard, Madison, WI (US)

(21) Appl. No.: 14/798,021

(22) Filed: Jul. 13, 2015

Related U.S. Application Data
(60) Provisional application No. 62/023,784, filed on Jul. 11, 2014.

(51) Int. Cl. C12N 15/10; C12Q 1/68; C12N 9/58

(52) U.S. Cl. CPC C12N 15/1013; C12N 9/58; C12Q 1/6806; C12Q 1/6844; C12Y 304/21064

(57) ABSTRACT

Provided herein is technology relating to isolating and/or extracting DNA from gastric fluid samples, e.g., pancreatic juice samples. In particular, the technology relates to methods, reagents, and kits for isolating DNA from stimulated pancreatic juice samples, for use, e.g., in methylation and mutation analyses.

This application claims priority to U.S. Provisional Application Ser. No. 62/023,784, filed Jul. 11, 2014, which is incorporated herein by reference.

### FIELD OF INVENTION

Provided herein is technology relating to isolating and/or extracting nucleic acid from biological samples. In particular, provided herein is technology relating to isolating and/or extracting DNA from, gastric fluid samples such as pancreatic effluent samples.

### BACKGROUND

Strikingly, 43,000 men and women are diagnosed each year with pancreatic cancer (PanC), which will cause nearly 37,000 deaths annually (Jemal et al. (2010) “Cancer statistics” CA Cancer J Clin 60: 277-300). As a result, PanC is the fourth leading cause of cancer deaths (id). Patients who present with symptoms typically already have advanced stage disease and only 15% meet criteria for potentially curative surgery (Ghaneh et al. (2007) “Biology and management of pancreatic cancer” Gut 56: 1134-52). Despite surgery, 85% will die of recurrent disease (Sohn et al. (2000) “Resected adenocarcinoma of the pancreas-616 patients: results, outcomes, and prognostic indicators” J Gastrointest Surg 4: 567-79). PanC mortality exceeds 95% and the 5-year survival rate is less than 25% for patients having curative surgery (Cleary et al (2004) “Prognostic factors in resected pancreatic adenocarcinoma: analysis of actual 5-year survivors” J Am Coll Surg 198: 722-31; Yeo et al (1995) “Pancreaticoduodenectomy for cancer of the head of the pancreas. 201 patients” Ann Surg 221: 721-33).

Among patients with syndromic predisposition to PanC or strong family history, aggressive, invasive screening strategies using computed tomography scans or endoscopic ultrasound have shown a 10% yield for neoplasia (Canto et al. (2006) “Screening for early pancreatic neoplasia in high-risk individuals: a prospective controlled study” Clin Gastroenterol Hepatol 4: 766-81). This screening strategy is impractical for the general population where most PanC arises without a known pre-disposition (Klein et al. (2001) “Familial pancreatic cancer” Cancer J 7: 266-73).

The nearly uniform lethality of PanC has generated intense interest in understanding pancreatic tumor biology. Precursor lesions have been identified, including pancreatic intraepithelial neoplasm (PanIN, grades I-III) and intraductal papillary mucinous neoplasm (IPMN) (Fernandez-del Castillo et al. (2010) “Intraductal papillary mucinous neoplasms of the pancreas” Gastroenterology 139: 708-13, 713.e1-2; Haugk (2010) “Pancreatic intraepithelial neoplasia—can we detect early pancreatic cancer?” Histopathology 57: 503-14). Study of both precursors and malignant lesions has identified a number of molecular characteristics at genetic, epigenetic, and proteomic levels that could be exploited for therapy or used as biomarkers for early detection and screening (Kaiser (2008) “Cancer genetics. A detailed genetic portrait of the deadliest human cancers” Science 321: 1280-1; Omura et al. (2009) “Epigenetics and epigenetic alterations in pancreatic cancer” Int J Clin Exp Pathol 2: 310-26; Tonack et al. (2009) “Pancreatic cancer: proteomic approaches to a challenging disease” Pancreatology 9: 567-76). Recent tumor and metastatic lesion mapping studies have shown that there may be a significant latency period between the development of malignant PanC and the development of metastatic disease, suggesting a wide window of opportunity for detection and curative treatment of presymptomatic earliest-stage lesions (Yachida et al. (2010) “Distant metastasis occurs late during the genetic evolution of pancreatic cancer” Nature 467: 1114-7).

The pancreas produces an effluent (“juice”) that can be collected for analysis, e.g. during endoscopic procedures. Pancreatic juice is a an alkaline liquid, typically clear, that contains a variety of enzymes, including deoxyribonuclease (DNase), ribonuclease (RNase), trypsinogen, chymotrypsinogen, elastase, carboxypeptidase, pancreatic lipase, and amylase, which aid in digestion that aid in the digestion of proteins, carbohydrates, fats, and nucleic acids.

Pancreatic cancers shed cells and DNA into pancreatic juice. However, extracting nucleic acid (such as DNA) from pancreatic juice for diagnostic purposes is complicated by the presence of the digestive enzymes and also by the presence of agents such as bile salts and blood that are known to inhibit diagnostic methods (e.g., PCR). Further, the types and amounts of inhibitors present in different samples can be highly variable sample-to-sample. Differences in nucleic acid yield and differences in the amounts and types of assay inhibitors carried though different nucleic acid purification procedures contribute to unacceptably high variation in nucleic acid detection assay results, frustrating attempts to develop detection assays that are sufficiently reliable for clinical diagnostic applications.

### SUMMARY OF THE INVENTION

The present invention relates to improved processes for extracting and/or isolating nucleic acid (e.g., DNA) from gastric samples, particularly from stimulated pancreatic juice and other types of gastric fluid. Indeed, experiments conducted during the course of developing embodiments for the present invention achieved improved DNA extraction (e.g., improved DNA percentage yield, improved consistency and reduced variability sample-to-sample) from pancreatic juice through utilization of a lysis buffer comprising a chaotropic agent and detergent, and in preferred embodiments, further comprising a pH neutralizing agent, and subsequent DNA extraction via a high-surface area solid support (e.g., mixable silica-coated magnetic beads). Embodiments of the invention provide markedly improved sample-to-sample consistency in the amount and quality of nucleic acid isolated from biological samples, e.g., pancreatic juice samples.

In some embodiments, the pancreatic fluid is diluted with an aqueous solution, e.g., water, buffer, or neutralizing solution, prior to a lysis step. For example, in some embodiments, the undiluted stimulated pancreatic effluent (e.g., collected as described in Example 1) is combined with water or buffer prior to addition of, for example, a protease such as proteinase K, or addition of a lysis and/or binding buffer. In some embodiments, the sample is exposed to a neutralizing reagent, e.g., potassium acetate and acetic acid. In some embodiments a neutralizing reagent is used prior to protease treatment, while in other embodiments, a neutralizing reagent is used in conjunction with or as part of a lysis reagent.

In some embodiments, a lysis buffer and a binding buffer may be used in separate steps. For example a lysis buffer comprising a chaotropic agent and a detergent may be added for a first treatment, and an additional chaotropic agent may be added prior to the addition of mixable particles. In other embodiments, a lysis buffer may also serve as a binding buffer. In certain embodiments, a lysis/binding buffer comprising guanidine HCl and a detergent, e.g., polyoxyethylene (20) sorbitan monolaurate (Tween 20™), may be used. In certain preferred embodiments, a lysis/binding buffer comprises about 7M guanidine HCl and about 20% polyoxyethylene (20) sorbitan monolaurate.

In some embodiments, a method of assaying DNA according to the invention comprises treating a sample with a protease; combining the sample treated with protease with a lysis reagent and with alcohol to form a sample mixture; adding mixable particles to the sample mixture under conditions wherein DNA in the sample binds to the mixable particles; washing the mixable particles bound to DNA; extracting bound DNA from the mixable particles; and performing a nucleic acid detection assay comprising DNA extracted from the mixable particles. In some embodiments, the protease comprises proteinase K, and in some embodiments, the lysis reagent comprises guanidine HCl and polyoxyethylene (20) sorbitan monolaurate. In certain preferred embodiments, the alcohol comprises isopropanol. In some embodiments, the nucleic acid detection assay comprises a flap endonuclease assay. In certain preferred embodiments, the nucleic acid detection assay comprises a polymerase chain reaction and a flap endonuclease assay (e.g., a QuARTS assay).

Additional embodiments will be apparent to persons skilled in the relevant art based on the teachings contained herein.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

The term “target,” when used in reference to a nucleic acid capture, detection, or analysis method, generally refers to a nucleic acid having a feature, e.g., a particular sequence of nucleotides to be detected or analyzed, e.g., in a sample suspected of containing the target nucleic acid. In some embodiments, a target is a nucleic acid having a particular sequence for which it is desirable to determine a mutation or methylation status. When used in reference to the polymerase chain reaction, “target” generally refers to the region of nucleic acid bounded by the primers used for polymerase chain reaction. Thus, the “target” is sought to be sorted out from other nucleic acid sequences that may be present in a sample. A “segment” is defined as a region of nucleic acid within the target sequence. The term “sample template” refers to nucleic acid originating from a sample that is analyzed for the presence of a target.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-S′.

Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The term “solid support” as used herein includes all the materials on which a target (e.g., DNA) can be immobilized. Natural or synthetic materials, which may or may not be chemically modified, can be used as a solid support, in particular polymers such as polyvinyl chloride, polyethylene, polystyrenes, polyacrylate or polyamide, or copolymers based on vinyl aromatic monomers, esters of unsaturated carboxylic acids, vinylidene chloride, dienes or compounds having nitrile functions (acrylonitrile); polymers of vinyl chloride and of propylene, polymers of vinyl chloride and vinyl acetate; copolymers based on styrenes or substituted derivatives of styrene; synthetic fibers, such as nylon; inorganic materials such as silica, glass, ceramic or quartz; latexes, magnetic particles; metal derivatives. Additional examples include, but are not limited to, a microtitration plate, a sheet, a cone, a tube, a well, beads (e.g., magnetic beads), particles or the like, or a flat support such as a silica or silicon wafer.

As used herein, the terms “magnetic particles” and “magnetic beads” are used interchangeably and refer to particles or beads that respond to a magnetic field. Typically, magnetic particles comprise materials that have no magnetic field but that form a magnetic dipole when exposed to a magnetic field, e.g., materials capable of being magnetized in the presence of a magnetic field but that are not themselves magnetic in the absence of such a field. The term “magnetic” as used in this context includes materials that are paramagnetic or superparamagnetic materials. The term “magnetic”, as used herein, also encompasses temporarily magnetic materials, such as ferromagnetic or ferrimagnetic materials with low Curie temperatures, provided that such temporarily magnetic materials are paramagnetic in the temperature range at which silica magnetic particles containing such materials are used according to the present methods to isolate biological materials. The term “mixable” as used in reference to particles or beads refers to particles that are in free form, i.e., that are not immobilized, e.g., in a column, but that can be added to a sample and distributed in the sample fluid by mixing action (e.g., vortexing, stirring, shaking, repeated pipetting, etc.).

As used herein, the term “kit” refers to any delivery system for delivering materials. In the context of nucleic acid purification systems and reaction assays, such delivery systems include systems that allow for the storage, transport, or delivery of reagents and devices (e.g., inhibitor adsorbents, particles, denaturants, oligonucleotides, spin filters etc. in the appropriate containers) and/or supporting materials (e.g., buffers, written instructions for performing a procedure, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contains a subportion of the total kit components. The containers may be delivered to the intended recipient together or separately. For example, a first container may contain an materials for sample collection and a buffer, while a second container contains capture oligonucleotides and denaturant. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes, including, e.g., polymerase chain reaction (PCR). See, e.g., amplification assays described in WO 2012/155072, which is incorporated herein by reference in its entirety.

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER invasive cleavage assay, Hologic, Inc.) and are described, e.g., in U.S. Pat. Nos. 5,846,717; 5,985,557; 5,994,069; 6,001,567; 6,090,543; and 6,872,816; Lyamichev et al., Nat. Biotech., 17:292 (1999), Hall et al., PNAS, USA, 97:8272 (2000), and US 2009/0253142, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Barnay Proc. Natl. Acad. Sci USA 88, 189-93 (1991)); and sandwich hybridization methods (e.g., U.S. Pat. No. 5,288,609, herein incorporated by reference in its entirety).

In some embodiments, target nucleic acid is amplified (e.g., by PCR) and amplified nucleic acid is detected simultaneously using an invasive cleavage assay. Assays configured for performing a detection assay (e.g., invasive cleavage assay) in combination with an amplification assay are described in US Patent Publication US 20090253142 A1 (App. Ser. No. 12/404,240), incorporated herein by reference in its entirety for all purposes. Additional amplification plus invasive cleavage detection configurations, termed the QUARTS method, are described in U.S. Pat. Nos. 8,361,720, 8,916,344, 8,715,937; and U.S. patent application Ser. No. 13/594,674, incorporated herein by reference in their entireties for all purposes. The term “invasive cleavage structure” as used herein refers to a cleavage structure comprising i) a target nucleic acid, ii) an upstream nucleic acid (e.g., an invasive or “INVADER” oligonucleotide), and iii) a downstream nucleic acid (e.g., a probe), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, e.g., as disclosed, for example, in U.S. Pat. No. 6,090,543, incorporated herein by reference in its entirety. In some embodiments, one or more of the nucleic acids may be attached to each other, e.g., through a covalent linkage such as nucleic acid stem-loop, or through a non-nucleic acid chemical linkage (e.g., a multi-carbon chain). As used herein, the term “flap endonuclease assay” includes “INVADER” invasive cleavage assays and QuARTS assays, as described above.

As used herein, a “remote sample” as used in some contexts relates to a sample indirectly collected from a site that is not the cell, tissue, or organ source of the sample. For instance, when sample material originating from the pancreas is assessed in a stool sample (e.g., not from a sample taken directly from a pancreas), the sample is a remote sample.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human.

As used herein, the term “lysis” refers to the disruption of a cell to release or expose the contents of the cell, e.g., nucleic acids, proteins, organelles, etc. A lysis buffer, for example, may contain one or more reagents suitable for opening a cell and/or for providing a suitable environment for molecules or other cellular components released from a cell upon lysis.

As used herein in reference to pancreatic juice sample, the term “neutralizing”, e.g., refers to adjusting the frequently alkaline pH of a pancreatic juice sample to have a pH of less than or equal to about pH 7.

As used herein, the term “stimulated pancreatic juice” refers to pancreatic effluent produced and collected from a subject after stimulation of the pancreas, e.g., by administration of secretin, e.g., synthetic human secretin, to a subject.

### DETAILED DESCRIPTION

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. Furthermore, one skilled in the art can readily appreciate that the specific sequences in which methods are presented and performed are illustrative and it is contemplated that the sequences can be varied and still remain within the spirit and scope of the various embodiments disclosed herein.

Provided herein is technology relating to isolating and/or extracting nucleic acid from biological samples, specifically samples of gastric fluids such as pancreatic effluent, or “pan juice.” Such methods are not limited to extracting DNA from a particular type of biological sample. In some embodiments, the biological sample is a gastric fluid sample. Examples of gastric fluid samples include, but are not limited to, gastric secretions, pancreatic juice, a gastrointestinal biopsy sample, micro-dissected cells from a gastrointestinal biopsy, gastrointestinal cells sloughed into the gastrointestinal lumen, and/or gastrointestinal cells recovered from stool. Such samples may originate from the upper gastrointestinal tract, the lower gastrointestinal tract, or comprise cells, tissues, and/or secretions from both the upper gastrointestinal tract and the lower gastrointestinal tract. The sample may include cells, secretions, or tissues from the liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva. In some embodiments, the sample comprises stool. In some embodiments, the biological sample is obtained from a subject (e.g., a human subject).

The methods described herein provide for a surprisingly effective and efficient extraction of nucleic acid (e.g., DNA) from complicated biological samples such as pancreatic juice, e.g., stimulated pancreatic juice, and other gastric fluid samples. In particular, the methods described herein provide improvements in both the gross amount of DNA recovered from pancreatic juice sample, but also provide improved sample-to-sample consistency in the amount and quality of DNA recovered, in comparison to prior art methods such as commercially available DNA extraction products commonly used for isolation of DNA from such samples. It was discovered that treatment of stimulated pancreatic juice samples containing DNA using lysis buffers and/or neutralizing reagents described herein, followed by binding the DNA to mixable particles rather than filters, membranes, or immobilized or packed particles, produced higher yields, and less sample-to-sample variability. The use of mixable magnetic particles is described, e.g., in U.S. Pat. No. 6,296,937, incorporated herein by reference in its entirety for all purposes, and are provided commercially, e.g., as “MagneSil” Paramagnetic Particles (catalogue number Z336D, Promega Corp., Madison, Wis.), and use of the mixable particles provides for more effective binding, washing, and eluting, relative to conventional technologies, e.g., mini-column methods in which the binding matrix is in a column, thereby limiting the exposure of the matrix to the sample fluid. Mixable particles are also amenable to automation of the methods provided herein. Specific embodiments of the technology are provided below.

In some embodiments, the techniques involve a lysis step utilizing a lysis buffer. In some embodiments, the techniques involve a nucleic acid extraction step utilizing a mixable particle, (e.g., magnetic beads). In some embodiments, the techniques involve performance of the lysis step followed by performance of the nucleic acid extraction step. In some embodiments, the lysis step involves exposure of a pan juice sample to a lysis buffer. Indeed, experiments conducted during the course of developing embodiments for the present invention demonstrated improved DNA extraction (e.g., higher DNA percentage yield, less variability) from a pancreatic juice sample utilizing a lysis buffer comprising a chaotropic agent (e.g., guanidine HCl), a detergent (e.g., 50% Tween-20™), and water in comparison to use of a commercially available lysis buffer (Buffer AL™; Qiagen).

The lysis buffer is not limited to utilizing a particular type of chaotropic agent. Generally, a chaotropic reagent is a substance which disrupts the three dimensional structure in macromolecules such as proteins, DNA, or RNA and denatures them. Chaotropic agents interfere with stabilizing intra-molecular interactions mediated by non-covalent forces such as hydrogen bonds, Van der Waals forces, and hydrophobic effects. Examples of applicable chaotropic agents include, but are not limited to, butanol, ethanol, guanidine thiocyanate, guanidinium chloride, lithium perchlorate, lithium acetate, magnesium chloride, phenol, propanol, sodium dodecyl sulfate, thiourea, and urea. The lysis buffer is not limited to utilizing a particular concentration for the chaotropic agent. In some embodiments, the chaotropic agent is guanidine HCl. In some embodiments, the concentration of guanidine HCl is between 0.1M-10.0M. In some embodiments, the guanidine HCl is approximately 7M guanidine HCl. In some embodiments, the guanidine HCl is approximately 3M guanidine HCl. In some embodiments a chaotropic solution comprises guanidine thiocyanate.

The lysis buffer is not limited to use of a particular detergent. In some embodiments, the detergent is a non-ionic detergent. In some embodiments, the detergent is a surfactant. In some embodiments, the detergent is a polysorbate surfactant. In some embodiments, the detergent is a polyoxyethylene derivative of sorbitan monolaurate. In some embodiments, the detergent is polyoxyethylene (20) sorbitan monolaurate (e.g., polysorbate 20 (Tween-20™)).

In some embodiments, the Tween-20™ is 50% Tween-20™. In some embodiments, the Tween-20™ is 20% Tween-20™. In some embodiments, the detergent is Nonidet P-40 (NP-40).

In some embodiments, the lysis step further comprises exposure of the sample to a protease, e.g., a serine protease. In some embodiments, the serine protease is proteinase K.

The lysis step is not limited to exposing a particular amount of serine protease to the biological sample. In some embodiments, for example, wherein the amount of biological sample is 100 μl, 20 μl (+/−10%) of serine protease (e.g., proteinase K solution) is exposed to the biological sample; in some embodiments, 10 μl (+/−10%) of serine protease (e.g., proteinase K) is exposed to the biological sample; in some embodiments, 30 μl (+/−10%) of serine protease (e.g., proteinase K) is exposed to the biological sample.

In some embodiments, the lysis step further comprises exposure of the biological sample to water. The lysis step is not limited to exposing a particular amount of water to the biological sample. In some embodiments, for example, wherein the amount of biological sample is 100 μl, 100 μl of water is exposed to the biological sample.

In some embodiments, the method comprises exposing a pan juice sample to a neutralization solution (e.g., a pH neutralization solution). Indeed, experiments conducted during the course of developing embodiments for the present invention demonstrated improved DNA extraction through utilization of various neutralization solutions (see, Example 6). Such methods are not limited to particular neutralization solutions. In some embodiments, the neutralization solution comprises water, potassium acetate, acetic acid and sodium azide (as described in Example 6). In some embodiments, the neutralization solution comprises NaH₂PO₄ and citric acid (e.g., 61.45 mM citric acid, 77.1 mM Na₂HPO₄, pH 4; or 17.65 mM citric acid, 164.7 mM Na₂HPO₄, pH 7). In certain embodiments, the sample is neutralized prior to treatment with a lysis buffer, as described above.

The technology further provides methods using mixable solid supports, e.g., particles, beads, etc. In some embodiments, the solid support includes magnetic beads. Indeed, in some embodiments, the magnetic particles are used for the treatment and isolation of DNA, e.g., particles comprising a magnetic core and a silica coating. The silica coating binds DNA and the magnetic core provides an efficient way to concentrate and isolate the particles (and bound DNA) using a magnet. In some embodiments, the silica-coated magnetic particles are MagneSil Paramagnetic Particles (Promega, Madison, Wis.; catalogue number AS 1220 or AS640A, promega.com).

The technology is not limited to any particular type of mixable magnetic bead or particles. Embodiments of the technology described herein make use of any magnetic beads (e.g., paramagnetic beads) that have an affinity for nucleic acids. In some embodiments, the magnetic beads have a magnetite (e.g., Fe₃O₄) core and a coating comprising silicon dioxide (SiO₂). The bead structure (e.g., size, porosity, shape) and composition of the solution in which a nucleic acid is bound to the bead can be altered to bind different types (e.g., DNA or RNA in single stranded, double stranded, or other forms or conformations; nucleic acids derived from a natural source, synthesized chemically, synthesized enzymatically (e.g., by PCR)) and sizes of nucleic acids (e.g., small oligomers, primers, genomic, plasmids, fragments (e.g., consisting of 200 or fewer bases) selectively. These characteristics of the beads affect the binding and elution of the nucleic acids to the beads. Related technologies are described, e.g., in U.S. Pat. Nos. 6,194,562; 6,270,970; 6,284,470; 6,368,800; 6,376,194, each incorporated herein by reference. Also contemplated are magnetic beads coated with, e.g., organosilane (as described in U.S. Pat. No. 4,554,088); carboxylated polyacrylate (as described in U.S. Pat. No. 5,648,124); cellulose (as described in U.S. patent application Ser. No. 10/955,974); hydroxysilane (as described in U.S. patent application Ser. No. 11/459,541); and hydrophobic aliphatic ligands (as described in U.S. patent application Ser. No. 12/221,750), each incorporated herein by reference for all purposes.

The technology is not limited to a particular size of magnetic bead or particle. Accordingly, embodiments of the technology use magnetic beads of a number of different sizes. Smaller beads provide more surface area (per weight unit basis) for adsorption, but smaller beads are limited in the amount of magnetic material that can be incorporated in the bead core relative to a larger bead. In some embodiments, the particles are distributed over a range of sizes with a defined average or median size appropriate for the technology for which the beads are used. In some embodiments, the particles are of a relatively narrow monodal particle size distribution.

In some embodiments, the beads that find use in the present technology have pores that are accessible from the exterior of the particle. Such pores have a controlled size range that is sufficiently large to admit a nucleic acid, e.g., a DNA fragment, into the interior of the particle and to bind to the interior surface of the pores. The pores are designed to provide a large surface area that is capable of binding a nucleic acid. Moreover, in one aspect the technology is not limited to any particular method of nucleic acid (e.g., DNA) binding and/or isolation. Thus, in some embodiments, aspects of the technology relating to the DNA extraction are combined with other suitable methods of DNA isolation (e.g., precipitation, column chromatography, etc.).

The beads (and bound material) are removed from a mixture using a magnetic field. In some embodiments, other forms of external force in addition to a magnetic field are used to isolate the biological target substance according to the present technology. For example, suitable additional forms of external force include, but are not limited to, gravity filtration, vacuum filtration, and centrifugation.

Embodiments of the technology apply an external magnetic field to remove the complex from the medium. In some embodiments, such a magnetic field is suitably generated in the medium using any one of different techniques. For example, in some embodiments, one positions a magnet on the outer surface of a container of a solution containing the beads, causing the particles to migrate through the solution and collect on the inner surface of the container adjacent to the magnet. The magnet is held in position on the outer surface of the container such that the particles are held in the container by the magnetic field generated by the magnet, while the solution is decanted out of the container and discarded. A second solution is added to the container, and the magnet removed so that the particles migrate into the second solution. Alternatively, a magnetizable probe is inserted into the solution and the probe magnetized, such that the particles deposit on the end of the probe immersed in the solution. The probe is removed from the solution, while remaining magnetized, immersed into a second solution, and the magnetic field discontinued permitting the particles go into the second solution. Commercial sources exist for magnets designed to be used in both types of magnetic removal and transfer techniques described in general terms above. See, e.g., MagneSphere Technology Magnetic Separation Stand or the PolyATract Series 9600™ Multi-Magnet, both available from Promega Corporation; Magnetight Separation Stand (Novagen, Madison, Wis.); or Dynal Magnetic Particle Concentrator (Dynal, Oslo, Norway). Some embodiments comprise use of a magnetic device according to U.S. Pat. Appl. Ser. No. 13/089,116, which is incorporated herein by reference in its entirety for all purposes. Furthermore, some embodiments contemplate the use of a “jet channel” or pipet tip magnet separation (e.g., as described in U.S. Pat. Nos. 5,647,994 and 5,702,950). Some embodiments contemplate the use of an immersed probe approach (e.g., as described in U.S. Pat. Nos. 6,447,729 and 6,448,092), e.g., as exemplified by the KingFisher systems commercially available from Thermo Scientific.

In some embodiments, the nucleic acid extraction techniques generally proceed as follows. First, the magnetic beads are washed in a binding buffer to remove storage and preservative solution. In a separate reaction, the biological sample is subject to a lysis step as described above. Next, the biological sample and a binding buffer comprising a chaotropic agent/protein denaturant (e.g., comprising 4.0-8.0 M guanidine thiocyanate (GTC), e.g., in some embodiments, approximately 4.8M GTC) are added to the beads and incubated to bind the DNA to the beads. In some embodiments, the bead washing and DNA binding steps are combined in a single step in which an excess amount of binding buffer is added to the beads followed by addition of the biological sample (e.g., having completed the lysis step). After binding, the DNA bound to the beads is exposed to a magnetic field (e.g., a magnet), and the fluid is removed. Next, the beads are washed, e.g., to remove residual sample contents, inhibitors, etc. In preferred embodiments, the DNA is then eluted in an appropriate DNA elution buffer such as 10 mM Tris-HCl (pH 8) and 0.1 mM EDTA.

In some embodiments, the present invention provides methods for performing extraction of DNA from a biological sample including the steps of: (i) lysis of a biological sample containing DNA (e.g., pancreatic juice) in a sample vessel with a lysis buffer comprising between 0.05 M and 10 M of a chaotropic agent (e.g., guanidine HCl, “GuHCl”); (ii) adding to the sample vessel magnetic beads and a binding buffer (e.g., comprising 4.0-8.0 M guanidine thiocyanate (GTC) or, in some embodiments, approximately 4.8M GTC) and incubating to bind the DNA to the beads; (iii) exposing the DNA bound to the beads to a magnetic field (e.g., a magnet) and removing a supernatant; and (iv) eluting the DNA in an appropriate DNA elution buffer.

In some embodiments, such methods further involve use of reagents necessary to amplify the eluted DNA (e.g., a PCR reaction using a specific pair of amplification primers) followed by amplification of the target DNA within the preparation (e.g., for detection of a specific target DNA within the biological sample).

In some embodiments, such methods are used in screening for any target nucleic acid of interest from a biological sample (e.g., a pancreatic juice sample). In some embodiments, the extraction methods of the present invention are used in screening for a neoplasm in a sample obtained from a subject. For example, in some embodiments, such methods comprise extracting DNA from a pancreatic juice sample obtained from a human subject, assaying a mutation state or methylation state of a marker in the sample (e.g., a pancreatic cancer marker as described in U.S. patent application Ser. No. 14/206,596, and International Patent Application Nos. PCT/US14/24582 and PCT/US14/24589); and identifying the subject as having a neoplasm when the mutation state or methylation state of a marker is different than a mutation state or methylation state of the marker assayed in a control sample, e.g., a sample from a subject that does not have a neoplasm. In some embodiments, the method further comprises locating the neoplasm site within the subject, wherein the methylation state of the marker indicates the neoplasm site within the subject.

In some embodiments, the sample vessel in which DNA is captured and washed is a well of a multi-well plate, e.g., a plate having 24, 96, 384, or 1536 wells, or any other number of wells. In some embodiments, the methods of the technology are performed in an automated process, e.g., using robotics and or automated liquid handling. In some embodiments, the DNA extraction is conducted on a microfluidic card or chip.

In some embodiments, the extracted DNA is quantified and analyzed. In some embodiments, the analysis comprises direct sequencing, pyrosequencing, methylation-sensitive single-strand conformation analysis (MS-SSCA), high resolution melting analysis, methylation-sensitive single-nucleotide primer extension (MS-SnuPE), base-specific cleavage/mass spectrometry (e.g., by MALDI-TOF), methylation-specific PCR (MSP), microarray analysis, restriction digest analysis, INVADER assay, combined bisulfite restriction analysis, or methylated DNA immunoprecipitation (MeDIP). These and other methods are reviewed in more detail in, e.g., Fraga M F & Esteller M (2002), “DNA methylation: a profile of methods and applications”, BioTechniques 33(3): 632, 634, 636-49; El-Maarri O (2003), “Methods: DNA methylation”, Advances in Experimental Medicine and Biology 544: 197-204; Laird P W (2003), “The power and the promise of DNA methylation markers”, Nat. Rev. Cancer 3(4): 253-66; Callinan P A & Feinberg A P (2006), “The emerging science of epigenomics”, Hum Mol Genet 15 (90001): R95-101, which are all incorporated by reference in their entireties for all purposes.

In some embodiments, a quantitative allele-specific real-time target and signal amplification (QuARTS) assay is used to evaluate a mutation or methylation state. Three reactions occur in each QUARTS assay, including amplification (reaction 1) and target probe cleavage (reaction 2) in the primary cleavage reaction; and FRET cassette cleavage (or secondary cleavage reaction) with fluorescent signal generation (reaction 3). When target nucleic acid is amplified with specific primers, a specific detection probe with a flap sequence loosely binds to the amplicon. The presence of the specific invasive oligonucleotide at the target binding site causes CLEAVASE enzyme to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between a fluorophore and quencher moieties on the FRET cassette, which produces a fluorescent signal. The cleavage reactions can cut multiple probes per target and release multiple fluorophores per flap, providing exponential signal amplification. The target amplification and signal amplification reactions can run concurrently, during the incubation and/or thermal cycling of the reaction mixture. QUARTS assays can detect multiple targets in a single reaction well by using FRET cassettes with different dyes. See, e.g., in Zou et al. (2010) “Sensitive quantification of methylated markers with a novel methylation specific technology” Clin Chem 56: A199; U.S. Pat. Nos. 8,361,720, 8,916,344, 8,715,937; and U.S. patent application Ser. No. 13/594,674.

In some embodiments, the methylation and/or mutation states of a plurality of markers are assayed. In some embodiments, assaying the state of the marker in the sample comprises determining the methylation/mutation state of one base, while in some embodiments, a plurality of bases are assayed. In some embodiments, a methylation state of the marker comprises an increased or decreased methylation of the marker relative to a normal methylation state of the marker. In some embodiments, the methylation state of the marker comprises a different pattern of methylation of the marker e.g., a different collection of particular methylated bases in the marker, relative to a normal methylation state of the marker.

The methods are not limited to analysis of a particular number of bases within a marker. In some embodiments, the marker is a region of 100 or fewer bases. In some embodiments, the marker is a region of 500 or fewer bases. In some embodiments, the marker is a region of 1000 or fewer bases. In some embodiments, the marker is a region of 5000 or fewer bases. In some embodiments, the marker is one base. In some embodiments, the marker is in a high CpG density promoter.

In some embodiments, the marker is a pancreatic cancer marker as described in U.S. patent application Ser. No. 14/206,596, and International Patent Application Nos. PCT/US14/24582 and PCT/US14/24589.

Such methods are not limited to a particular manner of assaying. In some embodiments, assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide.

In one aspect, the technology described herein is amenable to automation, e.g., processing without extensive or any human intervention, e.g., by robotics, computer-control, etc. As such, some embodiments relate to the use of a lysis buffer as described herein and solid support (e.g., magnetic beads) in an automated method or system for processing nucleic acids, e.g., extracting DNA from gastric fluid samples (e.g., pancreatic juice samples). In some embodiments, the processes comprise a heating step, e.g., a boiling step.

Experimental data collected during the development of the technology demonstrated that the technology described provides for the efficient recovery of DNA molecules from a pancreatic juice sample. Accordingly, embodiments of the technology provided herein relate to the purification and quantitative isolation (e.g., greater than 90% recovery, greater than 95% recovery, preferably greater than 97% recovery, and most preferably more than 99% recovery) of small nucleic acid (e.g., DNA) fragments. The technology comprises both the efficient capture of DNA by the beads and the efficient release of the isolated DNA from the beads, both under conditions manipulable by a user of the technology to effect, as desired, binding and release as appropriate for the application.

In some embodiments, the technology is related to compositions (e.g., reaction mixtures). In some embodiments, compositions comprising lysis buffers and biological samples (e.g., pancreatic juice) are provided. In some embodiments, compositions comprising lysis buffers and/or neutralization solutions are provided. In some embodiments, compositions comprising extracted DNA (e.g., DNA extracted from pancreatic juice) in combination with one or more PCR primers are provided. In some embodiments, the primers are mutation- or methylation-specific primers. In some embodiments, compositions comprising extracted DNA (e.g., DNA extracted from pancreatic juice) in combination with a bisulfite reagent are provided. Some embodiments provide a composition comprising extracted DNA (e.g., DNA extracted from pancreatic juice) and a methylation-sensitive restriction enzyme. Some embodiments provide a composition comprising extracted DNA (e.g., DNA extracted from pancreatic juice) and a polymerase. In some embodiments, an extracted DNA composition comprises a 5′ nuclease, e.g., a FEN-1 endonuclease, e.g., from an archaeal organism.

Further provided are kits for extracting nucleic acid (e.g., DNA) from pancreatic juice and/or a gastric fluid sample. In some embodiments, lysis buffers (e.g., lysis buffers containing guanidine HCl, detergent, and water) are provided. In some embodiments, a kit further comprises neutralizing agent(s). In some embodiments, the technology provides a kit comprising a lysis buffer (e.g., a lysis buffer containing guanidine HCl, detergent, and water), mixable magnetic particles, wash buffer(s), and/or an elution buffer. In some embodiments, the kits further provide the reagents sufficient, necessary, and/or useful to conduct a quantitative measurement and analysis of the extracted nucleic acid (e.g., DNA). In some embodiments, the kits further comprise positive/negative controls, sample collection devices, software (e.g., software for data analysis), proteases, etc.

In some embodiments, it is to be understood that one or more solutions of the kit are to be provided by the user of the kit. For example, in some embodiments a wash buffer is not included in the kit and is supplied by the user of the kit. In some embodiments, kits according to embodiments of the technology comprise a sample tube, an instruction for use, and packaging.

### EXPERIMENTAL EXAMPLES

The examples provided below illustrate certain aspect of the development of the technology of the invention.

### Example 1

### Collection of Secretin-Stimulated Pancreatic Juice

The pancreas of a subject may be stimulated to produce pancreatic fluids by a number of methods, including, e.g., having a subject chew gum, or by administration of a stimulant such as secretin. This example provides an exemplary method of producing and collecting stimulated pancreatic fluids for use in the methods of the present invention.

Human secretin is a gastrointestinal peptide hormone produced by cells in the duodenum in response to acidification. Human secretin is obtainable as a purified synthetic peptide with an amino acid sequence identical to the naturally occurring hormone. Synthetic human secretin is chemically defined as follows:

Molecular Weight 3039.44

Empirical Formula: C130H220N44O39

CAS #108153-74-8

Structural Formula: His-Ser-Asp-Gly-Thr-Phe-Thr-Ser-Glu-Leu-Ser-Arg-Leu-Arg-Glu-Gly-Ala-Arg-Leu-Gln-Arg-Leu-Leu-Gln-Gly-Leu-Val-NH₂ (ChiRhoStim® Human Secretin for Injection, package insert (2004), ChiRhoClin, Inc., Burtonsville, Md.).

Administration of secretin stimulates the production of pancreatic fluids, which are then collected. Secretin administration and effluent collection are done, e.g., as follows:

A preparation containing 16 μg of purified synthetic human secretin, 1.5 μg of L-cysteine hydrochloride, 20 μg of mannitol, and 0.9 μg of sodium chloride is dissolved in 8 ml of Sodium Chloride Injection USP, producing an injectable solution containing 2 μg/ml of synthetic human secretin.

Subjects fast overnight, or for about 12-15 hours. A double-lumen tube is passed through the mouth into the second portion of the duodenum, with the proximal lumen of the tube placed in the gastric antrum and the opening of the distal lumen just beyond the papilla of Vater.

Secretin may be administered before or after placement of the endoscope. See, e.g., Raimondo, et al., Clin. Gastroent. And Hepatology, v1:397-403 (2003) and ChiRhoStim package insert, supra). Secretin is administered intravenously at a dose of about 0.2 to 0.4 μg/kg of body weight, over the course of about 1 to 5 min.

Duodenal fluid is collected by suctioning as it is secreted, e.g., every 5-15 minutes over the course of 60 to 120 minutes. Samples of stimulated pancreatic juice may be stored on ice during collection and/or frozen at −80° C. for later use. (see, e.g., Raimondo, supra, ChiRhoStim, supra, and Fukashima, et al., Cancer Biology & Therapy, 2(1):78-83 (2003).

### Example 2

### Comparing Alternative Lysis Buffers and Comparing Mixable Particles Versus Spin-Filter Binding

This example compared DNA extraction from stimulated pancreatic juice samples utilizing a commercially available extraction technique employing spin filter binding (Qiagen QIAamp™) and a technique of the present invention comprising use of alternative lysis buffers and magnetic extraction using high-surface area mixable magnetic beads (referred to hereinbelow as the “PanExtract Method”). Improved DNA extraction was shown through use of the alternative lysis buffer in combination with the bead/magnetic extraction technique. Finally, assay of reference DNAs, e.g., beta-actin (ACTB) gene was used to determine the amount of DNA isolated using each procedure.

Two previously tested pancreatic juice samples were selected from samples received from the Mayo Clinic. Three 100 μl aliquots were taken from the sample and were extracted and purified as described below. All samples (5 μl) were analyzed using QuARTs flap endonuclease assay (described in U.S. Pat. Nos. 8,361,720 and 8,715,937 and U.S. patent application Ser. No. 12/946,745).

The lysis buffers tested were as follows:

### New Lysis Buffer 1 (LysB-1):

NaCl, 150 mM

NP-40 (IGEPAL CA-630), 1.0%

Tris-HCl, 50 mM (pH 8.0)
(For 200 ml of Buffer LysB-1, 6 ml of 5 M NaCl, 20 ml of 10% NP-40, 10 ml of 1 M Tris (pH 8.0), and 164 ml of H₂O are combined; stored at 4° C.)

### New Lysis Buffer 2 (LysB-2):

20% Tween 20

3M Guanidine HCl
(For 100 ml of Buffer LysB-2, 42.9 ml of 7M Guanidine HCl, 40 ml of 50% Tween-20, and 17.1 ml of H₂O are combined; stored at 4° C.)
   The DNA extraction method was as follows:

### PanExtract Method of Sample Prep:

Pipet 20 μl Proteinase K into the bottom of a 2.0 ml microcentrifuge tube (or well of multi-well plate).
Add 100 μl stimulated pancreatic juice sample to the microcentrifuge tube.
Add 100 μl H₂O to the microcentrifuge tube.
Add 200 μl of either Buffer AL (QIAamp lysis buffer; Qiagen), lysis buffer LysB-1 or lysis buffer LysB-2 to the sample.
Mix by pulse-vortexing for 15 seconds
Incubate at 56° C. for 10 minutes
Briefly centrifuge the 2.0 ml microcentrifuge tube to remove drops from the inside of the lid.
Add 200 μl ethanol (96-100%) to the sample, and mix again by pulse-vortexing for 15 sec. After mixing, briefly centrifuge the tube to remove drops from the inside of the lid.
Pipette the entire sample into a well of a 96-well Deep Well Plate (“DWP”).
Add 350 μl 4.8M Guanidine Thiocyanate (“GTC”).
Add 500 μl silica beads (800 μg of beads)
Incubate at 30° C. for 30 minutes with shaking.
Place tube(s) on magnet and let the beads collect. Aspirate and discard the supernatant.
Add 1000 μl wash buffer (10 mM Tris HCl, 80% EtOH) to the beads, and incubate at 30° C. for 3 minutes with shaking.
Place tubes on magnet and let the beads collect. Aspirate and discard the supernatant.
Add 500 μl wash buffer to the beads and incubate at 30° C. for 3 minutes with shaking.
Place tubes on magnet and let the beads collect. Aspirate and discard the supernatant.
Add 250 μl wash buffer and incubate at 30° C. for 3 minutes with shaking.
Place tubes on magnet and let the beads collect. Aspirate and discard the remaining buffer.
Dry the beads at 70° C. for 15 minutes, with shaking.
Add 70 μl elution buffer (10 mM Tris HCl, pH 8.0, 0.1 mM EDTA) to the beads and incubate at 65° C. for 25 minutes with shaking.
Place tubes on magnet and let the beads collect for 5 minutes.
Aspirate and transfer the supernatant to a new 1.5 ml tube.
Analyze all samples using QUARTS assays to determine DNA percentage yield.

### Comparative Method:

QIAamp Spin Column Method, using QIAamp buffers (see, e.g., “QIAamp DNA Mini and Blood Mini Handbook 06/2012” Qiagen Corp., incorporated herein by reference):
Pipet 20 μl Proteinase K into the bottom of a 2.0 ml microcentrifuge tube.
Add 100 μd Sample (pancreatic juice) to the microcentrifuge tube.
Add 100 μl H₂O to the microcentrifuge tube.
Add 200 μl lysis Buffer AL (Qiagen) to the sample. Mix by pulse-vortexing for 15 seconds
Incubate at 56° C. for 10 minutes
Briefly centrifuge the 2.0 ml microcentrifuge tube to remove drops from the inside of the lid.
Add 200 μl ethanol (96-100%) to the sample, and mix again by pulse-vortexing for 15 seconds. After mixing, briefly centrifuge the 1.5 ml microcentrifuge tube to remove drops from the inside of the lid.
Carefully apply the mixture to a QIAamp spin column (in a 2.0 ml collection tube), close the cap, and centrifuge at 6000×g (8000 rpm) for 1 minute. Place the QIAamp spin column in a clean 2.0 ml collection tube, and discard the tube containing the filtrate.
Add 500 μl wash Buffer AW1 (Qiagen) to the spin column. Close the cap and centrifuge at 6000×g (8000 rpm) for 1 minute. Place the QIAamp spin column in a clean 2 ml collection tube, and discard the collection tube containing the filtrate.
Add 500 μl wash Buffer AW2 (Qiagen) without wetting the rim. Close the cap and centrifuge at full speed (20,000×g; 14,000 rpm) for 3 minutes.
Place the QIAamp spin column in a clean 1.5 ml microcentrifuge tube, and discard the collection tube containing the filtrate. Carefully open the QIAamp spin column and add 100 μl elution Buffer AE (Qiagen). Incubate at room temperature for 1 min, and then centrifuge at 6000×g (8000 rpm) for 1 minute. Retain the eluate comprising the prepared DNA.

Table I shows the amount of DNA detected using each lysis buffer and method. These data show the increased yield of the ACTB target DNA with use of the LysB-2 lysis buffer in combination with magnetic beads, as compared to the QIAamp lysis buffer and spin filter binding technique. Results from using the QIAamp lysis buffer AL with the beads in place of the spin filter are also shown. Together these data show that use of LysB-2 buffer, the buffer containing the chaotropic agent, and use of mixable beads for binding both contribute to the improved recovery of the target DNA from samples tested.

TABLE 1

| | | | ACTB Fold Increase |
|---|---|---|---|
| | | Lysis | Compared to Spin |
| | Method | Buffer | Filter |
| Sample #76 | Spin Filter | AL | 1.0 |
| | Magnetic Beads | AL | 4.0 |
| | Magnetic Beads | LysB-1 | 0.7 |
| | Magnetic Beads | LysB-2 | 4.8 |
| Sample #78 | Spin Filter | AL | 1.0 |
| | Magnetic Beads | AL | 2.7 |
| | Magnetic Beads | LysB-1 | 0.0 |
| | Magnetic Beads | LysB-2 | 2.8 |

### Example 3

### Comparison of Lysis Buffers and Capture Method on DNA Isolation from Different Samples

Stimulated pancreatic fluids upon collection present a variety of different colors, e.g., clear, black, yellow, green, indicating different patient conditions, salts, other solutes and materials in the sample. The pancreatic juice samples listed in Tables 2A-2H, having the recited sample colors, were selected based on the known presence of ACTB target DNA strands, as described above. Aliquots of either 100 μl or 200 μl were taken from the samples and DNA was extracted and purified as described below. All samples were analyzed using the QuARTs assay. All experimental conditions were compared with the Qiagen spin column extraction method (Qiagen QIAamp™), as described above.

The PanExtract method was performed as described above on 100 μl and 200 μl aliquots of sample, using either lysis Buffer AL (Qiagen) or lysis buffer LysB-2, using the PanExtract Method Sample Prep as described in Example 2. The comparative examples were prepared using the QIAamp spin column procedure as described in Example 2. For both procedures, the 100 μl sample aliquots were brought to 200 μl volume by addition of 100 μl of H₂O to the proteinase K mixture. All samples were analyzed using QUART'S assays directed to ACTB to determine DNA percentage yield.

Tables 2A-H show the increase in assay signal from detectable target DNA (ACTB) with use of the LysB-2 lysis buffer and magnetic bead binding method, in comparison to QIAamp spin filter binding technique and AL lysis buffer, with results reported along with the color of the original pancreatic juice sample.

TABLE 2A

| | | | | Fold Improvement |
|---|---|---|---|---|
| Sample | | | | in detected |
| ID; | Sample | | | DNA relative to |
| vol | color: | Lysis Buffer | Platform | Spin Filter |
| 28 | Dark | | | |
| | green | | | |
| 100 μl | | AL | Magnetic Beads | 4.39 |
| 100 μl | | LysB-2 | Magnetic Beads | 4.71 |
| 100 μl | | AL | Spin Filter | |
| 200 μl | | AL | Magnetic Beads | 4.67 |
| 200 μl | | LysB-2 | Magnetic Beads | 4.51 |
| 200 μl | | AL | Spin Filter | |

TABLE 2B

| | | | | Fold Improvement |
|---|---|---|---|---|
| Sample ID; | Sample | Lysis | | relative to |
| vol | color | Buffer | Platform | Spin Filter |
| 34 | black | | | |
| 100 μl | | AL | Magnetic Beads | 68.93 |
| 100 μl | | LysB-2 | Magnetic Beads | 63.40 |
| 100 μl | | AL | Spin Filter | |
| 200 μl | | AL | Magnetic Beads | 47.64 |
| 200 μl | | LysB-2 | Magnetic Beads | 64.77 |
| 200 μl | | AL | Spin Filter | |

TABLE 2C

| | | | | Fold Improvement |
|---|---|---|---|---|
| Sample ID; | Sample | Lysis | | relative to |
| vol | color | Buffer | Platform | Spin Filter |
| 42 | yellow | | | |
| 100 μl | | AL | Magnetic Beads | 24.03 |
| 100 μl | | LysB-2 | Magnetic Beads | 20.73 |
| 100 μl | | AL | Spin Filter | |
| 200 μl | | AL | Magnetic Beads | 17.22 |
| 200 μl | | LysB-2 | Magnetic Beads | 16.72 |
| 200 μl | | AL | Spin Filter | |

TABLE 2D

| | | | | Fold Improvement |
|---|---|---|---|---|
| Sample ID; | Sample | Lysis | | relative to |
| vol | color | Buffer | Platform | Spin Filter |
| 82 | red | | | |
| 100 μl | | AL | Magnetic Beads | 0.93 |
| 100 μl | | LysB-2 | Magnetic Beads | 0.83 |
| 100 μl | | AL | Spin Filter | |
| 200 μl | | AL | Magnetic Beads | 0.78 |
| 200 μl | | LysB-2 | Magnetic Beads | 1.28 |
| 200 μl | | AL | Spin Filter | |

TABLE 2E

| | | | | Fold Improvement |
|---|---|---|---|---|
| Sample ID; | | Lysis | | relative to |
| vol | Sample color | Buffer | Platform | Spin Filter |
| 17 | dark yellow | | | |
| 100 μl | | AL | Beads | 32.67 |
| 100 μl | | LysB-2 | Beads | 37.16 |
| 100 μl | | AL | Spin Filter | |
| 200 μl | | AL | Beads | 35.57 |
| 200 μl | | LysB-2 | Beads | 21.21 |
| 200 μl | | AL | Spin Filter | |

TABLE 2F

| | | | | Fold Improvement |
|---|---|---|---|---|
| Sample ID; | | Lysis | | relative to |
| vol | Sample color | Buffer | Platform | Spin Filter |
| 18 | Clear | | | |
| 100 μl | | AL | Beads | 2.44 |
| 100 μl | | LysB-2 | Beads | 3.38 |
| 100 μl | | AL | Spin Filter | |
| 200 μl | | AL | Beads | 1.95 |
| 200 μl | | LysB-2 | Beads | 3.01 |
| 200 μl | | AL | Spin Filter | |

TABLE 2G

| | | | | Fold Improvement |
|---|---|---|---|---|
| Sample ID; | | Lysis | | relative to |
| vol | Sample color | Buffer | Platform | Spin Filter |
| 27 | Clear | | | |
| 100 μl | | AL | Beads | 4.72 |
| 100 μl | | LysB-2 | Beads | 5.78 |
| 100 μl | | AL | Spin Filter | |
| 200 μl | | AL | Beads | 3.62 |
| 200 μl | | LysB-2 | Beads | 4.21 |
| 200 μl | | AL | Spin Filter | |

TABLE 2H

| | | | | Fold Improvement |
|---|---|---|---|---|
| Sample ID; | | Lysis | | relative to |
| vol | Sample color | Buffer | Platform | Spin Filter |
| 6 | Brown | | | |
| 100 μl | | AL | Beads | 19.67 |
| 100 μl | | LysB-2 | Beads | 23.32 |
| 100 μl | | AL | Spin Filter | |
| 200 μl | | AL | Beads | 25.45 |
| 200 μl | | LysB-2 | Beads | 25.73 |
| 200 μl | | AL | Spin Filter | |

These data show the effect of sample quality, as represented by sample color, on variability of performance of different DNA isolation buffers and binding matrices. These data also show that the LysB-2 lysis buffer gave consistently better results than the AL lysis buffer, and that, regardless of the lysis buffer used, use of the mixable beads substantially improved the yield of detectable DNA strands relative to the yield obtained using the spin filtration procedure.

### Example 4

### The Effect of pH and pH Neutralization on Strand Recovery from LysB-2 Lysis Buffer/Bead-Based Binding Method

This example characterized the percent recovery by the PanExtract DNA extraction method e.g., the in relation to known input strands of control plasmid DNA. This method further incorporates a neutralization step to control the pH of the extraction. This example demonstrates high percentage recovery through utilization of the neutralization buffer.

The pHs of the samples were determined to be as follows:

| Sample # | Sample Color | Sample pH |
|---|---|---|
| 001 | Brown | 5.5 |
| 023 | Clear | 9 |
| 087 | Yellow | 8.5 |
| 121 | Clear | 9 |
| 133 | Clear | 9 |
| 134 | Clear | 9 |
| 143 | Clear | 9 |
| 007 | Dark Yellow | 9 |
| 014 | Dark Yellow | 9 |
| 087 | Dark Yellow | 8.5 |
| 136 | Dark Yellow | 8 |
| 011 | white pale | 9 |
| | yellow | |
| 075 | white red | 8.5 |
| 143 | white | 9 |
| 121 | white | 9 |
| 140 | brown black | 8.5 |
| 039 | dark yellow | 8 |
| 113 | dark yellow | 7.5 |
| 151 | brown pale | 7.5 |
| | yellow | |

Two negative pan juice samples (#001 m pH 5.5 and #23, pH 9) were used to test the effects of neutralizing the pH. The two samples, 100 μl total sample volume, were spiked with 5e4 copies of control DNA (CLEC, CD1D, ACTB). Two replicates of each sample were used for a total of 8 samples in this experiment.

The protocol without neutralization buffer was performed using the LysB-2 lysis buffer as described in Example 2.

A neutralization solution was formulated as follows:

Neutralization Solution (NEU SLN) Formulation (pH 5)

| Final Conc. | Units | Component |
|---|---|---|
| N/A | N/A | Water |
| 150 | mM | Potassium Acetate |
| 80 | mM | Acetic Acid |
| 0.05 | % | Sodium Azide |

The protocol implementing neutralization was conducted as follows:

### Sample Preparation:

Thaw one vial of each of the negative pancreatic juice samples (lacking target nucleic acids).
Remove four 90 μl aliquots from each of the two Negative pan juice samples and place into separate wells of a 96-well Deep Well Plate (DWP).

### PanExtract Method Sample Prep:

Add 100 μl H₂O or Neutralization Solution (NEU SLN) to the samples in the DWP.
Add 200 μl of Buffer LysB-2 to the samples in the DWP.
Mix by pipetting up and down 3 times.
Add 10 μl of target DNA (5e3 strands/μl) (CLEC, CD1D and ACTB) into the appropriate (+) wells for both pan juice Samples.
Add 10 μl of Fish DNA diluent into each of the (−) Neg wells for both pan juice samples.
Add 200 μL of ethanol (96-100%) to each sample in the DWP.
Cover with sealing foil.
Shake for 30 seconds at 1100 rpm in the shaking incubator at Room Temperature.
Perform DNA extraction.
Proceed to QuARTs Setup.

AU samples were analyzed using the QUARTS assay method to determine DNA percentage yield.

The concentrations of the plasmid stocks (see, e.g., U.S. Patent Application Ser. No. 61/899,302, incorporated herein by reference) used to spike the reactions and the expected output (strand recovery count) are as shown in Table 6A.

| | NDRG4 | BMP3 | ACTB |
|---|---|---|---|
| | strands | strands | strands |
| Spike | | | |
| 5e4 Stock | 44682 | 47605 | 64833 |
| Expected | | | |
| Output | | | |
| 5e4 Stock | 6383 | 6801 | 9262 |
| | | CD1D | ACTB |
| | CLEC strands | strands | strands |
| Spike | | | |
| 5e4 Stock | 51757 | 45325 | 60691 |
| Expected | | | |
| Output | | | |
| 5e4 Stock | 7394 | 6475 | 8670 |

Tables 6B and 6C show the DNA strands recovered and the percentage yield (NDRG4, BMP3, ACTB; Table 6B) (CLEC, CD1D and ACTB; Table 6C) using the LysB-2 lysis buffer, a Neutralization Buffer (NEU SLN), and the magnetic beads-binding protocol.

TABLE 6B

| | | | | | | Plasmid Recovery | | |
|---|---|---|---|---|---|---|---|---|
| | | | NDRG4 | BMP3 | ACTB | % | % | % |
| Sample | Spike | Additive | strands | strands | strands | NDRG4 | BMP3 | ACTB |
| #001 | 0 | H2O | 0 | 0 | 0 | 0% | 0% | 0% |
| #001 | 5.00E+4 | H2O | 5627 | 6124 | 7633 | 88% | 90% | 82% |
| #001 | 5.00E+4 | NEU SLN | 5279 | 6127 | 5791 | 83% | 90% | 63% |
| #001 | 5.00E+4 | NEU SLN | 5165 | 6079 | 5526 | 81% | 89% | 60% |
| #023 | 0.00E+0 | H2O | 0 | 0 | 0 | 0% | 0% | 0% |
| #023 | 5.00E+4 | H2O | 91 | 110 | 737 | 1% | 2% | 8% |
| #023 | 5.00E+4 | NEU SLN | 1116 | 1380 | 3795 | 17% | 20% | 41% |
| #023 | 5.00E+4 | NEU SLN | 863 | 1133 | 3749 | 14% | 17% | 40% |

TABLE 6C

| | | | | | | Plasmid Recovery | | |
|---|---|---|---|---|---|---|---|---|
| | | | CLEC | CD1D | ACTB | % | % | % |
| Sample | Spike | Additive | strands | strands | strands | CLEC | CD1D | ACTB |
| #001 | 0 | H2O | 0 | 0 | 0 | 0% | 0% | 0% |
| #001 | 5.00E+4 | H2O | 6991 | 6184 | 7606 | 95% | 96% | 88% |
| #001 | 5.00E+4 | NEU SLN | 6197 | 5696 | 6180 | 84% | 88% | 71% |
| #001 | 5.00E+4 | NEU SLN | 6014 | 5611 | 5747 | 81% | 87% | 66% |
| #023 | 0.00E+0 | H2O | 0 | 0 | 0 | 0% | 0% | 0% |
| #023 | 5.00E+4 | H2O | 208 | 96 | 760 | 3% | 1% | 9% |
| #023 | 5.00E+4 | NEU SLN | 3113 | 1632 | 4202 | 42% | 25% | 48% |
| #023 | 5.00E+4 | NEU SLN | 2591 | 1302 | 3970 | 35% | 20% | 46% |

As seen from these data, samples having a low pH (e.g., sample 1) are not affected by the use of a neutralizing buffer, but recovery of the added DNA from alkaline samples (e.g., sample #23) may be remarkably improved, e.g., 5 to 20-fold.

### Example 5

### The Effect of Detergent Concentration on Strand Recovery from a Lysis Buffer/Mixable Bead-Based Binding Method

This example characterized the effects of detergent concentration on strand recovery. Pancreatic juice samples of different colors were pooled to produce a single sample type for testing. All experimental conditions were compared with the Qiagen spin column extraction method (Qiagen QIAamp™), as described above.

Protocol:

### I) Sample Pool

Aliquots of the following pancreatic juice samples (shown by sample ID and color) were pooled.
55 (dark yellow),
60 (brown black),
35 (clear),
65 (light brown),
58 (pale yellow),
62 (pale yellow),
63 (yellow)

### II) Lysis Buffer Conditions

The following lysis conditions were tested:

| Condition | Lysis Buffer |
|---|---|
| 1 | 300 μl 7M GuHCL with 15% Tween-20 |
| 2 | 300 μl 7M GuHCL with 20% Tween-20 |
| 3 | 300 μl 7M GuHCL with 25% Tween-20 |

### III) Pancreatic Juice Extraction Protocol

1. Add 50 μl Proteinase K (10 μg/ml, Promega Corp., Madison, Wis.) to a mixture of 200 μl of pooled pancreatic juice and 100 μl of water in a deep well plate. Incubate at room temperature for 2 minutes.
2. Add 300 μl of lysis buffer (see Conditions 1-3, above) and mix by pipetting up and down. Incubate for 10 minutes at 55° C. while mixing on a plate shaker.
3. Add 50 μl magnetic silica beads (800 μg of beads) and mix.
4. Add 350 μl isopropanol and mix up and down.
5. Incubate at 30° C. for 30 minutes with shaking, e.g., at 1200 rpm.
6. Place tube(s) on magnet and let the beads collect. Aspirate and discard the supernatant.
7. Add 1000 μl of wash buffer (10 mM Tris HCl (pH 8.0), 80% EtOH) to the beads and incubate at 30° C. for 3 minutes with shaking.
8. Place tube(s) on magnet and let the beads collect Aspirate and discard the supernatant.
9. Add 500 μl of wash buffer (10 mM Tris HCl (pH 8.0), 80% EtOH) and incubate at 30° C. for 3 minutes with shaking.
10. Place tube(s) on magnet and let the beads collect. Aspirate and discard the supernatant.
11. Add 250 μl of wash buffer (10 mM Tris HCl (pH 8.0), 80% EtOH) and incubate at 30° C. for 3 minutes with shaking.
12. Place tube(s) on magnet and let the beads collect. Aspirate and discard the supernatant.
13. Dry the beads at 70° C., 15 minutes, with shaking.
14. Add 70 μl of elution buffer (10 mM Tris-Cl (pH 8.0), 0.1M EDTA), incubate at 65° C. for 25 minutes with shaking.
15. Place tube(s) on magnet and let the beads collect for 5 minutes.
16. Aspirate and transfer the supernatant to a new tube or well of a sample plate.
17. Proceed with KRAS assay flap endonuclease assay with ACTB control.

All samples were analyzed using the QuARTs flap endonuclease assay as described in U.S. Pat. No. 9,000,146, which is incorporated herein by reference. KRAS1=FAM dye channel=34T, 35T, 38A. KRAS2=HEX dye channel=34A, 35A, 34C, 35C.

| Condition ID | KRAS 1 | KRAS 2 | ACTB |
|---|---|---|---|
| 1 | 58 | 139 | 93487 |
| 2 | 42 | 130 | 99484 |
| 3 | 31 | 80 | 65078 |
| Qiagen Control-1 | 47 | 96 | 31375 |

These data show that use of 20% Tween-20 is the optimal amount for maximum strand recovery in a GuHCl lysis buffer. Higher amounts appear to result in low strand recovery while lower amounts can somewhat decrease strand recovery. (see, e.g., the ACTB control).

### Example 6

### Exemplary Pancreatic Juice Extraction Protocol

In some embodiments, extraction of DNA from pancreatic juice (e.g., stimulated pancreatic fluid) proceeds generally as follows:
1. Combine:
   a. 200 μl Pancreatic juice (collected, e.g., as in Example 1)
   b. 100 μl water
   c. 50 μl Proteinase K (10 μg/ml)
in a microcentrifuge tube or well, e.g., of a deep well plate. Incubate at room temperature for 5 minutes.
2. Add 300 μl lysis buffer (3M GuHCl, 20% Tween) and 350 μl isopropanol to the sample and mix up and down. Incubate 10 minutes at 55° C. while mixing, e.g., on a plate shaker set to 950 RPM.
3. Add 50 μl magnetic silica beads (800 μg of beads) and mix, e.g., by pipetting up and down.
4. Incubate 30° C. for 30 minutes with shaking, e.g., at 1200 rpm.
5. Place tube(s) on magnet and let the beads collect. Aspirate and discard the supernatant.
6. Add 1000 μl wash buffer (10 mM Tris MCI pH 8.0, 80% EtOH) to the beads and incubate at 30° C. for 3 minutes with shaking.
7. Place tube(s) on magnet and let the beads collect. Aspirate and discard the supernatant.
8. Add 500 μl wash buffer (10 mM Tris HO pH 8.0, 80% EtOH) and incubate at 30° C. for 3 minutes with shaking.
9. Place tube(s) on magnet and let the beads collect. Aspirate and discard the supernatant.
10. Add 250 μl wash buffer (10 mM Tris HCl pH 8.0, 80% EtOH) and incubate at 30° C. for 3 minutes with shaking.
11. Place tube(s) on magnet and let the beads collect. Aspirate and discard the supernatant.
12. Dry the beads at 70° C., 15 minutes, with shaking.
13. Add 70 μl of elution buffer (10 mM Tris (pH 8.0) 0.1M EDTA), incubate 65° C. for 25 minutes with shaking
14. Place tube(s) on magnet and let the beads collect for 5 minutes.
15. Aspirate and transfer the supernatant to a new tube or well of a sample plate.
16. Proceed to nucleic acid detection assay (e.g., mutation detection assay and/or to bisulfite conversion and methylation detection assay.)
   One or more of the steps are optionally performed on an automated station, e.g., a Hamilton MicroLab STAR system.

### Example 7

### Sensitive DNA Marker Panel for Detection of Pancreatic Cancer by Assay of DNA in Stimulated Pancreatic Juice

It has been found that specific methylated DNA markers in pancreatic effluents can discriminate pancreatic cancer from chronic pancreatitis and normal pancreas (Gastroenterology 2013; 144:S-90). DNA for testing may be prepared according to the methods of the present invention.

Experiments are performed to compare methylated DNA markers and mutant KRAS assayed alone and combined in stimulated pancreatic juice to distinguish pancreatic cancer (PanC) samples from chronic pancreatitis (CP) and reference controls (CON). See, e.g., PCT Application Ser. No. PCT/US14/24589 (WO 2014/159652), and U.S. application Ser. No. 13/594,674, filed Aug. 8, 2012, each of which is incorporated by reference herein in its entirety.

On DNA extracted from 200 μl of stimulated pancreatic juice (freshly collected or stored, e.g., at −80° C.), gene methylation is determined after bisulfite treatment by quantitative allele-specific real-time target and signal amplification (QuARTS) for assay of ADCY1, CD1D, BMP3, PRKCB, KCNK12, C13ORF18, IKZF1, CLEC11A, TWIST1, NDRG4, ELMO, and 55957. Mutant KRAS mutations and the ACTB marker for total human DNA are also assayed using the QuARTS assay. From quantitative data, an algorithm is followed to achieve optimal discrimination by a panel combining all markers. At respective specificity cutoffs of 90% and 95%: the combined marker panel achieves highest PanC sensitivities.

All publications, patent applications, and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in oncology, pharmacology, biochemistry, medical science, or related fields are intended to be within the scope of the following claims.

We claim:

1. A method of treating a sample of stimulated pancreatic juice from a subject, comprising:
a) treating said sample with protease;
b) combining the sample treated with protease with a lysis reagent and an alcohol, wherein said lysis reagent comprises a chaotropic agent and a detergent;
c) adding mixable particles to said sample under conditions wherein DNA in the sample binds to said mixable particles, and
d) extracting bound DNA from said mixable particles.

2. The method of claim 1, comprising treating said sample with a neutralizing reagent.

3. The method of claim 2, wherein said neutralizing reagent comprises potassium acetate and acetic acid.

4. The method of claim 1, wherein said chaotropic agent comprises guanidine HCl and/or guanidine thiocyanate.

5. The method of claim 1, wherein said detergent comprises polyoxyethylene (20) sorbitan monolaurate and/or Nonidet-P40 (NP40).

6. The method of claim 1, wherein said lysis reagent comprises guanidine HCl and polyoxyethylene (20) sorbitan monolaurate.

7. The method of claim 1, wherein said protease comprises proteinase K.

8. The method of claim 1, wherein said alcohol comprised isopropanol and/or ethanol.

9. The method of claim 1, wherein said mixable particles comprise silica-coated magnetic particles.

10. A method of assaying DNA in a sample of stimulated pancreatic juice from a subject, the method comprising:
a) treating said sample with proteinase K;
b) combining the sample treated with proteinase K with a lysis reagent comprising guanidine HCl and polyoxyethylene (20) sorbitan monolaurate, and with isopropanol to form a sample mixture,
c) adding mixable particles to said sample mixture under conditions wherein DNA in the sample binds to said mixable particles;
d) separating mixable particles bound to DNA from said sample mixture;
e) washing said mixable particles bound to DNA;
f) extracting bound DNA from said mixable particles; and
g) performing a nucleic acid detection assay comprising DNA extracted from said mixable particles.

11. The method of claim 10, wherein said sample is exposed to a neutralizing reagent.

12. The method of claim 10, wherein said lysis reagent comprises about 7M guanidine HCl and 20% polyoxyethylene (20) sorbitan monolaurate.

13. The method of claim 10, wherein said washing comprises exposing said mixable particles bound to DNA to a wash buffer comprising Tris-HCl and ethanol.

14. The method claim 10, wherein said extracting said bound DNA from said mixable particles comprises exposing said mixable particles bound to DNA to an elution buffer comprising Tris-HCl and EDTA.

15. A kit for extracting DNA from stimulated pancreatic juice, comprising:
a) a protease;
b) a lysis reagent comprising a first chaotropic agent and a detergent;
c) a solution comprising a neutralizing reagent;
c) mixable particles comprising a silica surface; and
d) an elution solution.

16. The kit of claim 15, wherein said neutralizing reagent comprises potassium acetate and acetic acid.

17. The kit of claim 15, further comprising one or more of:
a sample vessel;
an alcohol;
a second chaotropic agent;
a wash buffer; and
a magnet.

18. The kit of claim 17, wherein said first and second chaotropic agents are the same or different, and wherein said first and said second chaotropic agents are selected from guanidine HCl and guanidine thiocyanate.

19. The kit of claim 17, wherein said alcohol comprises ethanol and/or isopropanol.

20. The kit of claim 15, wherein the detergent comprises polyoxyethylene (20) sorbitan monolaurate and/or Nonidet-P40 (NP40).

21. The kit of claim 15, wherein said mixable particles are silica coated magnetic particles.
